(19) United States
(12) Patent Application Publication
(10) Pub. No.: US 2024/0350445 A1
(43) Pub. Date: Oct. 24, 2024

(54) ANTI-FUNGAL COMPOSITIONS

(71) Applicant: BIOCIDIUM IP HOLDCO, CO., North Vancouver (CA)

(72) Inventors: Jan Willem TOEBES, Abbotsford (CA); Nicholas Philip SWAIN, North Vancouver (CA)

(21) Appl. No.: 18/681,326

(22) PCT Filed: Aug. 3, 2022
(86) PCT No.: PCT/CA2022/051183

§ 371 Date: Feb. 5, 2024

Related U.S. Application Data
(60) Provisional application No. 63/228,953, filed on Aug. 3, 2021.

(51) Int. Cl. A61K 31/201; A01N 33/04; A01N 33/08; A01N 37/06; A01N 37/36; A01P 3/00; A61K 9/00; A61K 31/131; A61K 31/132; A61K 31/133; A61K 31/19; A61P 31/10

(52) U.S. Cl. CPC A61K 31/201; A01N 33/04; A01N 33/08; A01N 37/06; A01N 37/36; A01P 3/00; A61K 9/0014; A61K 31/131; A61K 31/132; A61K 31/133; A61K 31/19; A61P 31/10

(57) ABSTRACT

The present invention provides anti-fungal compositions comprising at least one unsaturated fatty acid or a pharmaceutically acceptable salt thereof: at least one alpha-hydroxy acid or a pharmaceutically acceptable salt thereof; and at least one amino alcohol.

### FIELD OF THE INVENTION

This invention pertains to the field of anti-fungal compositions and, in particular, to anti-fungal compositions comprising one or more fatty acids, one or more hydroxy acids and one or more amines.

### BACKGROUND OF THE INVENTION

Fungal infections are becoming a major health concern for a number of reasons, including the limited number of anti-fungal agents currently available, the increasing incidence of species resistant to older anti-fungal agents, and the growing population of immuno-compromised patients at risk for opportunistic fungal infections. The most common clinical fungal isolate is Candida albicans (comprising about 19% of all isolates). In one study, nearly 40% of all deaths from hospital-acquired infections were due to fungi (Sternberg, Science, 1994, 266:1632-1634).

Thus, new anti-fungal agents/compositions are needed to address both the growing resistance to present therapies and the general lack of efficacy of existing anti-fungal agents.

In the cosmetics and food industry also there is a constant need for agents having anti-fungal properties, in particular for the preservation of products which are otherwise perishable, but also for direct cosmetic or therapeutic treatment of fungi which can have an adverse influence on the human or animal body. Reference may be made by way of example to fungi which can cause athlete's foot, ringworm, and candidiasis (thrush) and others.

There remains a need for anti-fungal compounds and/or compositions that have improved and/or broad spectrum anti-fungal activity.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

It is an object of the present invention to provide a composition having improved anti-fungal activity. In accordance with an aspect of the present invention, there is provided an anti-fungal composition comprising at least one unsaturated fatty acid having 6 to 16 carbon atoms or a pharmaceutically acceptable salt thereof, at least one alpha-hydroxy acid or a pharmaceutically acceptable salt thereof; and at least one amino alcohol.

In accordance with another aspect of the present invention, there is provided a pharmaceutical formulation comprising a composition as defined above, and a pharmaceutically acceptable carrier.

In accordance with another aspect of the present invention, there is provided a use of the composition or the pharmaceutical formulation of the present invention for inhibiting growth and/or proliferation of fungi.

In accordance with another aspect of the present invention, there is provided a method of killing and/or inhibiting the growth of fungi comprising applying an effective amount of the anti-fungal composition or the formulation as defined above.

### BRIEF DESCRIPTION OF THE DRAWINGS

These and other features of the invention will become more apparent in the following detailed description in which reference is made to the appended drawings.

FIG. 1 presents the Initial Populations (CFU/mL) and the post-exposure Recovery Populations (Log 10) of each challenge species, as well as the Percent and Log 10 Reductions produced by the Composition A at each time of exposure.

FIG. 2 illustrates results of a disk assay demonstrating anti-fungal activity of composition A against Aspergillus niger (10⁵ cfu/g), Rhodotrula glutenis (10⁵ cfu/g) and Penicillum spp (10⁵ cfu/g). A. untreated, B. treated.

FIG. 3 illustrates results of a disk assay demonstrating anti-fungal activity of known anti-fungal agents against Aspergillus niger, Rhodotrula glutenis and Penicillum spp. A. untreated, B. treated with combined 2-methyl-4isothiazolin-3-one (0.5 wt %) and 1,2-benzisothiazol-3 (3H)-one (0.5 wt %). C. 5-choloro-2-methyl-4-isothiazolin-3-one (0.5 wt %).

FIG. 4 illustrates results of 17-day topical treatment of degu mouse with mycosis, left panel shows pre-treatment, right panel shows mouse after 17-days of treatment.

### DETAILED DESCRIPTION OF THE INVENTION

Unless defined otherwise, all technical and scientific terms used herein have the same meaning as commonly understood by one of ordinary skill in the art to which this invention pertains.

The present invention provides novel anti-fungal compositions and uses thereof. In the context of the present invention the term “anti-fungal activity” refers to anti-anti-fungal/yeast activity also referred to as “antimycotic”.

The term “anti-fungal” includes fungicidal, i.e., the killing of fungi and/or fungistatic, inhibitory to growth or proliferation of fungi.

As used herein, the term “alkyl” refers to a straight chain or branched, alkyl group of one to ten carbon atoms. This term is further exemplified by such groups as methyl, ethyl, n-propyl, i-propyl, n-butyl, t-butyl, I-butyl (or 2-methylpropyl), and the like.

As used herein, the term “amine” refers a compound having formula NRR′R″, where R, R′ and R″ may independently be hydrogen, alkyl, or alkyl substituted with one or more substituents such as OH, NH₂.

As used herein, the term “hydroxyalkyl” refers to an alkyl group substituted with at least a hydroxyl group. This term is further exemplified by such groups as hydroxymethyl, 2-hydroxyethyl, 2-hydroxybutyl, 4-hydroxybutyl, 2,3-dihydroxypropyl, etc.

The terms “therapy” and “treatment,” are used interchangeably herein, refer to an intervention performed used to treat and/or prevent fungal infection, also called mycosis.

The terms “subject” or “patient,” as used herein, refer to an animal in need of treatment, including humans and other mammals.

As used herein, the term “skin” is according to its conventional meaning, namely an external organ including the epithelial tissue. The term “mucosa” is also used with its usual meaning, which relates to all the mucosal barriers in the body, such as the gastrointestinal, pulmonary, sublingual, buccal, rectal, vaginal, nasal, urethral and ocular barriers.

Administration of the composition of the present invention “in combination with” one or more additional active agents, is intended to include simultaneous (concurrent) administration and consecutive administration. Consecutive administration is intended to encompass various orders of administration of the additional agent(s) and the compositions of the present invention. The additional agent, in a preferred embodiment, is an anti-fungal and/or anti-bacterial agent.

As used herein, the term “about” refers to approximately a +/−10% variation from a given value. It is to be understood that such a variation is always included in any given value provided herein, whether or not it is specifically referred to.

### The Compositions

The present invention provides an anti-fungal composition comprising a combination of at least one unsaturated fatty acid or a pharmaceutically acceptable salt thereof; at least one alpha-hydroxy carboxylic acid or a pharmaceutically acceptable salt thereof; and at least one amine. The amine can be an amino alcohol.

The unsaturated fatty acids suitable for the use in the composition of the present invention have from 6 to 16 carbon atoms, preferably the fatty acid has from 8 to 12 carbon atoms. In one embodiment, the free fatty acid is undecylenic acid.

The alpha hydroxy acids of the present invention can be selected from glycolic acid, lactic acid, citric acid, mandelic acid, oxalic acid, and malonic acid. In one embodiment, the alpha hydroxy acid is lactic acid.

Nonlimiting examples of amines suitable for the use in the composition of the present invention include ethylenediamine, N,N,-dimethylbutylamine, triethylamine, etc.

In some embodiments, the amine suitable for the use in the composition of the present invention is an amino alcohol having a formula:

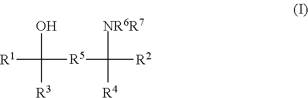

wherein R¹ and R³ are each independently H, alkyl, R² and R⁴ are each independently H, alkyl, or hydroxyalkyl; R⁵ is absent or is a C₁-C₆ alkylene; and R⁶ and R⁷ are independently H, C₁-C₆ alkyl or hydroxyalkyl.

In one embodiment, in the formula (I) above, R⁵ is absent, R² and R⁴ are both C₁-C₆ alkyl.

In one embodiment, in the formula (I) above, R⁵ is absent, R² is C₁-C₆ alkyl and R⁴ is CH₂OH.

In one embodiment, the amino alcohol of formula (I) is amino methyl propane diol (AMPD).

In one embodiment, the amino alcohol of formula (I) is mono-, di- or tri-C₁-C₆alkanol amine. In one embodiment, the amino alcohol is monoethanolamine (MEA). In one embodiment, the amino alcohol is triethanolamine (TEA).

Non-limiting examples of other amino alcohols include, 2-amino-2-methyl-1-propanol, 2-amino-2-methyl-1,3-propanediol, 2-amino-2-ethyl-1,3-propanediol,2-dimethylamino-2-methylpropanol, dimethylglucamine, n-butyldiethanolamine, and 2-butylamino ethanol.

In one embodiment, the amino alcohol is 95% 2-amino-2-methyl-1-propanol in an aqueous solution.

The individual concentrations of unsaturated fatty acid, alpha hydroxy acid and amino alcohol can be in the range of about 5% to about 90% by weight of the total weight of the composition.

In one embodiment, in the composition of the present invention, the concentration of the unsaturated fatty acid is about 5%, 10%, 15%, 20%, 25%, 30%, 35%, 40%, 45%, 50%, 55%, 60%, 65%, 70%, 75%, 80%, 85%, 90% or a percentage between any two of these values.

In one embodiment, in the composition of the present invention, the concentration of the alpha hydroxy acid is about 5%, 10%, 15%, 20%, 25%, 30%, 235%, 40%, 45%, 50%, 55%, 60%, 65%, 70%, 75%, 80%, 85%, 90% or a percentage between any two of these values.

In one embodiment, in the composition of the present invention, the concentration of the amino alcohol is about 5%, 10%, 15%, 20%, 25%, 30%, 35%, 40%, 45%, 50%, 55%, 60%, 65%, 70%, 75%, 80%, 85%, 90% or a percentage between any two of these values.

In one embodiment, in the composition of the present invention, the amount of unsaturated fatty acid is in the range of about 40% to about 80% by weight of the total weight of the composition, the amount of alpha hydroxy acid is in the range of about 10% to about 40% by weight of the total weight of the composition, and the amount of amino alcohol is in the range of about 5% to about 25% by weight of the total weight of the composition. In one embodiment, in the composition of the present invention the amount of unsaturated fatty acid is in the range of about 50% to about 70% by weight of the total weight of the composition, the amount of alpha hydroxy acid is in the range of about 20% to about 35% by weight of the total weight of the composition, and the amount of amino alcohol is in the range of about 10% to about 20% by weight of the total weight of the composition. In one embodiment, the amount of unsaturated fatty acid is in the range of about 55% to about 65% by weight of the total weight of the composition, in the composition of the present invention the amount of alpha hydroxy acid is in the range of about 25% to about 30% by weight of the total weight of the composition, and the amount of amino alcohol is in the range of about 10% to about 15% by weight of the total weight of the composition.

In one embodiment, the composition of the present invention comprises about unsaturated fatty acid 58.00 wt. %, about 28.00 wt. % of alpha hydroxy acid, and about 14.00 wt. % of amino alcohol.

The anti-fungal composition may further comprise at least one viscosity-enhancing agent, i.e., thickening agent. Preferably the viscosity-enhancing agent is selected from xanthan gum, alginic acid, agar, carrageenan, locust bean gum, pectin, cellulose derivatives, gelatin and combinations thereof.

The anti-fungal composition may further comprise at least one emulsifying agent, such as polysorbate (Tween) 20, polysorbate 40, polysorbate 60, polysorbate 80, polyoxyethylene glycol alkyl ethers, glucoside alkyl ethers, polyoxyethylene glycol octylphenol ethers, polyoxyethylene glycol alkylphenol ethers, glycerol alkyl esters, poloxamers, polyoxyl castor oil, and combinations thereof. In one embodiment, the emulsifying agent is polysorbate 80.

The compositions of the present invention can be used neat/undiluted or formulated as a solution, emulsion or dispersion in a suitable liquid medium.

Non-limiting examples of a liquid medium include water, alcohols or other organic solvents (optionally mixed with water), oil, etc.

In some embodiments, the anti-fungal composition is formulated as a suspension or dispersion in water.

Non-limiting examples of suitable oils include, peanut oil, olive oil, sesame oil or coconut oil, and the like or mineral oils such as liquid paraffin. The oily suspensions may contain a thickening agent, for example, beeswax, hard paraffin or cetyl alcohol.

The amount of anti-fungal composition provided in the solution, emulsion or dispersion can be about 0.1% to 5% by weight of the formulation.

### Uses of the Anti-Fungal Compositions

The present invention provides for the use of the compositions disclosed herein for the inhibition, prevention or eradication of the growth and/or proliferation of fungi, either alone or in combination with known anti-microbial agents and/or anti-fungal agents.

In one embodiment, the present invention provides a method of inhibiting fungal growth by contacting a fungus with an effective amount of the compositions disclosed herein either alone or in combination with one or more other anti-fungal agents. Representative examples of fungi that may be inhibited with the compositions include, but are not limited to, Trichophyton (e.g. T. mentagrophytes, T. rubrum), Histoplasma (e.g. H. capsulstum), Coccidioides, Blastomyces, Paracoccidioides, Cryptococcus (e.g. C. neoformans), Aspergillus (e.g. A. fumigatus, A. flaws, A. niger, A. nidulans, A. terreus, A. sydowii, A. flavatus, and A. glaucus), Zygomycetes (e.g. Basidiobolus, Conidiobolus, Rhizopus, Mucor, Absidia, Mortierella, Cunninghamella, and Saksenaea), Candida (e.g. C. albicans, C. tropicalis, C. parapsilosis, C. stellatoidea, C. krusei, C. parakrusei, C lusitaniae, C. pseudorropicalis, C. guilliermondi and C. glabrata), Cryptosporidium parvum, Sporothrix schenckii, Piedraia hortae, Trichosporon beigelii, Malassezia furfur, Phialophora verrucosa, Fonsecae pedrosoi, Madurella mycetomatis and Pneumocystis carinii.

In one embodiment, the present invention provides a formulation containing a composition as defined herein for external use as a pharmaceutically acceptable skin cleanser. In one embodiment, the compositions of the present invention can also be used as dermocosmetic compositions.

The compositions of the present invention can also be included as an active ingredient in personal care and household products/formulations, fabric, paper products, plastic products, and the like.

Non-limiting examples of personal care products/formulations include soaps, personal cleansers, sprays, deodorants, shampoos, mouthwashes, toothpastes, and the like.

Non-limiting examples of house hold products/formulations include polishes, paints, detergents, household cleansers, and the like. Such household cleansers, sprays or soaps can also be used in hospital settings for the prevention of nosocomial infections.

The anti-fungal composition of the present invention may be incorporated into a personal care and household formulations using techniques known in the art. Thus, the anti-fungal composition may be added to the personal care or household products/formulations as a solution, emulsion or dispersion in a suitable liquid medium. Alternatively, the anti-fungal agent may be added, in an undiluted form or may be added with a solid carrier or diluent. The anti-fungal composition may be added to the pre-formed personal care/household formulation or may be added during the formation of the personal care/household formulation, either separately or premixed with one or more of the other components of the formulation.

The anti-fungal composition of the present invention can generally be used in quantities of between 0.1% and 20% by weight of the personal care/household formulations. In one embodiment, the amount of anti-fungal composition is between 0.1% and 5% by weight of the formulation. In one embodiment the amount of anti-fungal composition is about 0.3% to about 5% by weight of the formulation.

The compositions may be formulated for application to surfaces to inhibit the growth of a fungal species thereon, for example, hard surfaces, such as countertops, desks, chairs, laboratory benches, tables, floors, sinks, showers, toilets, bathtubs, bed stands, tools or equipment, doorknobs, windows, porous surfaces, such as towels, sheets and other bed linen, washcloths, sponges or other cleaning articles.

In addition, the invention contemplates the use of the compositions in formulations to kill or inhibit the growth of fungal species in food preparations, or to sterilise surgical and other medical equipment and implantable devices, including prosthetic joints. The compositions can also be formulated for use in the in situ sterilisation of indwelling invasive devices such as intravenous lines and catheters, which are often foci of infection.

### Pharmaceutical Formulations and Administration of Anti-Fungal Compositions

For use as therapeutic agents in the treatment of fungal infections, or disorders or diseases associated therewith in a subject, the anti-fungal compositions of the present invention are typically formulated prior to administration. Therefore, the present invention provides pharmaceutical formulations comprising one or more compositions of the present invention and a pharmaceutically-acceptable carrier, diluent, or excipient. The present pharmaceutical formulations are prepared by standard procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container.

The pharmaceutical formulations comprising the anti-fungal compositions according to the present invention may be formulated in a number of ways depending upon the desired treatment and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration.

For administration to an individual for the treatment of an infection or disease, the present invention also contemplates the formulation of the pharmaceutical formulations comprising the anti-fungal composition into oral dosage forms such as tablets, capsules and the like. For this purpose, the composition can be combined with conventional carriers, such as magnesium carbonate, magnesium stearate, talc, sugar, lactose, pectin, dextrin, starch, gelatine, tragacanth, methylcellulose, sodium carboxymethyl-cellulose, low melting wax, cocoa butter and the like. Diluents, flavouring agents, solubilizers, lubricants, suspending agents, binders, tablet-disintegrating agents and the like can also be employed, if required. The anti-fungal compositions can be encapsulated with or without other carriers. In accordance with the present invention, the proportion of anti-fungal composition(s) in any solid and liquid formulation will be at least sufficient to impart the desired activity to the individual being treated upon oral administration. The present invention further contemplates parenteral injection of the anti-fungal compositions, in which case the compositions are formulated as a sterile solution containing other solutes, for example, enough saline or glucose to make the solution isotonic.

For administration by inhalation or insufflation, the anti-fungal compositions can be formulated into an aqueous or partially aqueous solution, which can then be utilized in the form of an aerosol. Aqueous formulations of the anti-fungal compositions of the present invention may also be used in the form of ear or eye drops, or ophthalmic solutions. The present invention further contemplates topical use of the anti-fungal compositions. For this purpose they can be formulated as dusting powders, creams or lotions in pharmaceutically acceptable vehicles, which are applied to affected portions of the skin.

Formulations intended for oral use may be prepared according to procedures known in the art for the manufacture of pharmaceutical formulations and such formulations may further contain one or more sweetening agents, flavouring agents, colouring agents, preserving agents, or a combination thereof, in order to provide pharmaceutically elegant and palatable preparations. Tablets typically contain the anti-fungal composition(s) in admixture with non-toxic pharmaceutically acceptable excipients suitable for the manufacture of tablets, such as inert diluents, for example, calcium carbonate, sodium carbonate, lactose, calcium phosphate or sodium phosphate; granulating and disintegrating agents, for example, corn starch, or alginic acid; binding agents, for example, starch, gelatine or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatine capsules wherein the anti-fungal composition(s) is mixed with an inert solid diluent, for example, calcium carbonate, calcium phosphate or kaolin, or as soft gelatine capsules wherein the active ingredient is mixed with water or an oil medium, for example, peanut oil, liquid paraffin or olive oil.

Aqueous suspensions typically contain the anti-fungal composition(s) in admixture with excipients suitable for the manufacture of aqueous suspensions, such as suspending agents (for example, sodium carboxylmethylcellulose, methyl cellulose, hydropropylmethyl cellulose, sodium alginate, polyvinylpyrrolidone, gum tragacanth and gum acacia); dispersing or wetting agents such as a naturally-occurring phosphatide (for example, lecithin), or condensation products of an alkylene oxide with fatty acids (for example, polyoxyethylene stearate), or condensation products of ethylene oxide with long chain aliphatic alcohols (for example, hepta-decaethyleneoxycetanol), or condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example, polyethylene sorbitan monooleate). The aqueous suspensions may further contain one or more preservatives, for example, ethyl, or n-propyl-p-hydroxy benzoate; one or more colouring agents; one or more flavouring agents, or one or more sweetening agents, such as sucrose or saccharin, or a combination thereof.

Oily suspensions may be formulated by suspending the anti-fungal composition(s) in a vegetable oil, for example, peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the anti-fungal composition in admixture with a dispersing or wetting agent, suspending agent and one or more preservatives. Suitable dispersing or wetting agents and suspending agents are exemplified by those mentioned above. Additional excipients, for example, sweetening, flavouring and colouring agents, may also be present.

Pharmaceutical formulations of the present invention may also be in the form of oil-in-water emulsions. The oil phase may be a vegetable oil, for example, olive oil or peanut oil, or a mineral oil, for example, liquid paraffin, or mixtures thereof. Suitable emulsifying agents may be naturally-occurring gums (for example, gum acacia or gum tragacanth); naturally-occurring phosphatides (for example, soy bean lecithin), and esters or partial esters derived from fatty acids and hexitol anhydrides (for example, sorbitan monooleate), and condensation products of the partial esters with ethylene oxide (for example, polyoxyethylene sorbitan monooleate). The emulsions may also contain sweetening and flavouring agents.

Syrups and elixirs may be formulated with sweetening agents, for example, glycerol, propylene glycol, sorbitol or sucrose. Such formulations may also contain one or more demulcents, preservatives or flavouring and colouring agents, or combinations thereof.

The pharmaceutical formulations may be in the form of a sterile injectable aqueous or oleaginous suspension. This suspension may be formulated according to known art using suitable dispersing or wetting agents and suspending agents as described above. The sterile injectable preparation may also be a solution or a suspension in a non-toxic, parentally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Typically, a bland fixed oil is employed for this purpose such as a synthetic mono- or diglyceride. In addition, fatty acids such as oleic acid find use in the preparation of injectables. Adjuvants, such as local anaesthetics, preservatives and buffering agents, may also be included in the injectable formulation.

The composition(s) of the present invention may be administered, together or separately, in the form of suppositories for rectal or vaginal administration of the composition. These formulations can be prepared by mixing the composition with a suitable non-irritating excipient which is solid at ordinary temperatures but liquid at the rectal/vaginal temperature and will therefore melt to release the composition. Examples of such materials include cocoa butter and polyethylene glycols.

Another formulation of the present invention employs transdermal delivery devices (“patches”). Such transdermal patches may be used to provide continuous or discontinuous administration/application of the anti-fungal compositions of the present invention in controlled amounts. The construction and use of transdermal patches for the delivery of pharmaceutical agents is well known in the art (see, for example, U.S. Pat. No. 5,023,252; issued Jun. 11, 1991, incorporated herein by reference in its entirety). Such patches may be constructed for continuous, pulsatile, or on demand delivery of pharmaceutical agents.

In one embodiment, the composition(s) of the present invention can be incorporated into medical dressings such as Tegaderm pad from 3M (which act as traditional sponge gauze, a fungal barrier just helping to reduce the risk of infection). In Tegaderm structure, the main biocompatible wound dressing part is made of cellulose paper fibre coated with silicone material, and all supporting and adhering parts are made of synthetic materials such as: polyethylene, polyurethane, polyester and acrylate polymer.

It may be desirable or necessary to introduce the pharmaceutical formulations to the brain, either directly or indirectly. Direct techniques usually involve placement of a drug delivery catheter into the host's ventricular system to bypass the blood-brain barrier. An example of such an implantable delivery system, used for the transport of biological factors to specific anatomical regions of the body, is described in U.S. Pat. No. 5,011,472, incorporated herein by reference in its entirety.

The dosage of the anti-fungal composition to be administered is not subject to defined limits, but will usually be an effective amount. In general, the dosage will be the equivalent, on a molar basis, of the pharmacologically active free form produced from a dosage formulation upon the metabolic release of the active free drug to achieve its desired pharmacological and physiological effects. The pharmaceutical compositions are typically formulated in a unit dosage form, each dosage containing from, for example, about 0.05 to about 100 mg of the anti-fungal composition. The term “unit dosage form” refers to physically discrete units suitable as unitary dosages for administration to human subjects and other animals, each unit containing a predetermined quantity of anti-fungal composition calculated to produce the desired therapeutic effect, in association with a suitable pharmaceutical excipient.

Typical daily dosages of the anti-fungal compositions fall within the range of about 0.01 to about 200 mg/kg of body weight in single or divided dose. However, it will be understood that the amount of the composition actually administered will be determined by a physician, in the light of the relevant circumstances, including the condition to be treated, the chosen route of administration, the actual composition administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, for example, by first dividing larger doses into several smaller doses for administration throughout the day.

Compositions of the present invention formulated for topical administration are suitable in the treatment and/or prevention of fungal infections of the skin and mucosa.

Topical pharmaceutical and/or dermo-cosmetic formulations according to the invention comprise the anti-fungal composition mixed with one or more suitable excipients and may be, for example, in the form of cream, lotion, ointment, gel, gum, toothpaste, mouthwash or shampoo.

The pharmaceutical formulations and/or the dermocosmetic formulations can comprise about 0.1% to about 20% by weight of the anti-fungal composition of the present invention. In one embodiment, the amount of anti-fungal composition is about 0.1% to about 10% by weight of the formulation. In one embodiment, the amount of anti-fungal composition is about 0.1% to about 5% by weight of the formulation. In one embodiment the amount of anti-fungal composition is about 0.3% to about 2% by weight of the formulation.

Examples of suitable excipients that may be used in the formulation comprising the compositions according to the invention are solvents, diluents, gliding agents, preservatives, gums, sweeteners, coating agents, binders, disintegrating agents, lubricants, suspending agents, dispersing agents, colorants, flavouring agents, non-stick agents, surfactants, plasticisers, emulsifiers, chelating agents and emollients.

The solvent preferably used is water, but alcohols or other organic solvents may also be used, possibly mixed with water.

The choice of excipients is part of the normal knowledge of one skilled in the art, and will mainly depend on the pharmaceutical and/or dermocosmetic form chosen.

For example, a cream can be prepared by incorporating the anti-fungal composition of the present invention in a topical carrier consisting of liquid paraffin, dispersed in an aqueous medium by means of lubricants. An ointment can be prepared by mixing TSP with a topical carrier such as mineral oil or wax. A gel can be prepared by mixing TSP with a topical carrier containing a gelling agent.

The pharmaceutical and/or dermocosmetic composition according to the invention may also be a woven or non-woven material coated and/or impregnated with a mixture of the anti-fungal composition with a suitable carrier or a matrix in which the anti-fungal composition is dispersed so that it comes into contact with the skin for transdermal administration. Specific examples are bandage, gauze, towelettes, etc.

The choice of type of pharmaceutical and/or dermocosmetic form will depend mainly on the area to be treated and is part of the normal knowledge of one skilled in the art. For example, a gum or mouthwash may be more suitable to treat the oral cavity, whereas a cream, ointment, lotion or towelettes may be suitable for the skin of the face.

The formulations comprising the compositions according to the invention are preferably applied by topical administration directly to the area of the skin or mucosa which presents, or is assumed to present, a fungal infection or other disorders caused by the presence of microbes. The infection often originates in a part of the skin or mucosa which presents a lesion, such as a wound, laceration or burn. In such case, the formulations can be applied directly to the lesion and/or the surrounding area.

The compositions of the present invention can also be used in the treatment and/or prevention of several disorders of the skin and mucosa, which are known to be caused by bacteria, for example, psoriasis, eczema, acne, etc. Other treatments may include wound care, and burn care, etc.

### Anti-Fungal Activity of Compositions

The anti-fungal activity of a candidate composition can be tested using standard techniques known in the art. As is known in the art, anti-fungal activity of a composition may result in the killing of fungal cells (i.e. fungicidal activity), or it may result in the slowing or arrest of the growth of fungal cells (i.e. fungistatic activity). Thus the compositions may be fungicidal and/or fungistatic. Compositions of the present invention that slow or arrest fungal cell growth may be useful in combination treatments with other known anti-microbial agents.

To gain a better understanding of the invention described herein, the following examples are set forth. It should be understood that these examples are for illustrative purposes only. Therefore they should not limit the scope of the invention in any way.

### EXAMPLES

### Example 1: Anti-Fungal Compositions

Exemplary compositions of the present invention were prepared by mixing about 58.00 wt. % unsaturated fatty acid, about 28.00 wt. % of alpha hydroxy acid, and about 14.00 wt. % of one or more amines.

An exemplary composition of the present invention (composition 1) was prepared by mixing about 525 grams of undecylenic acid, about 250 grams of lactic acid, and about 125 grams of AMP-95%.

Composition 1 is soluble in most solvents (i.e., acetone, chloroform, methanol, ethanol, benzene, DMAC, DMSO) with no loss of bioactivity and can be solubilized in aqueous medium using a co-solvent system. Physical properties of the composition remained the same after heating (no color change or gelation or evaporation was observed).

Variations of Composition 1 were prepared by replacing AMP-95 with other amines:
Composition 2 (amine: ethylenediamine);
Composition 3 (amine: N,N,-dimethylbutylamine);
Composition 4 (amine: triethylamine);
Composition 5 (amine: ethanol Amine);
Composition 6 (amine: triethanolamine);
Composition 7 (amine: 2-dimethylamino-2-methylpropanol) (DMAMP-80 or DMAMP
Ultra PC);
Composition 8 (amine: 2-amino-2-ethyl-1,3-propanediol, aka AEPD VOX 1000);
Composition 9 (amine: 2-amino-2-methyl-1,3-propanediol);
Composition 10 (amine: Dimethylglucamine); and
Composition 11 [amine: 2-butylamino ethanol (aka Advantex)]

### Example 2: Aqueous Formulations Comprising Anti-Fungal Compositions

Aqueous formulations comprising Composition 1 were prepared by mixing composition 1 and triethanol amine (as dispersion agent) in a weight ratio of from 3.5:1.5 to 1.5:1, and the mixture was then diluted with water to achieve desired concentration of the composition in the final aqueous formulation.

An exemplary aqueous formulation of the present invention (Formulation 1) was prepared by mixing 60% by weight composition 1 and 40% by weight of triethanolamine, and diluting the mixture with water to have about 0.5 wt % Composition 1 in the final aqueous formulation.

Another exemplary aqueous formulation of the present invention (Formulation 2) was prepared by mixing 60% by weight composition 1 and 40% by weight of triethanolamine, and diluting the mixture with water to have about 1.0 wt % Composition 1 in the final aqueous formulation.

### Example 3: Evaluation of In Vitro Anti-Fungal Activity

The anti-fungal properties of exemplary “Composition 1” were evaluated against 19 fungal species (including yeast species) using an in-vitro time-kill method. The in-vitro time-kill evaluation of Composition 1 was performed versus suspensions of 19 fungal species, each in duplicate. The percent and log₁₀ reduction in the fungal population of each challenge strain was determined following exposure to the undiluted composition 1 for 1 minute, 3 minutes, 5 minutes, and 20 minutes. All agar-plating was performed in duplicate.

Fungal species tested were:
Candida auris (AR-Bank #0385),
Candida auris (AR-Bank #0389),
Candida auris (AR-Bank #0390),
Alternaria atra (ATCC #18040),
Aspergillus brasiliensis (ATCC #16404),
Aspergillus flavus (ATCC #9643),
Aureobasidium pullulans (ATCC #15233),
Cladosporium cladosporioides (ATCC #16022),
Epidermophyton floccosum (ATCC #52061),
Microsporum canis (ATCC #36299),
Penicillium citrinum (ATCC #9849),
Penicillium rubens (ATCC #11709),
Phoma pomorum (ATCC #MYA-3682),
Rhodotorula mucilaginosa (ATCC #66034),
Sporobolomyces roseus (ATCC #24257),
Stachybotrys chart arum (ATCC #201867),
Trichoderma virens (ATCC #9645),
Trichophyton interdigitate (ATCC #9533),
Trichophyton rubrum (ATCC #28188).

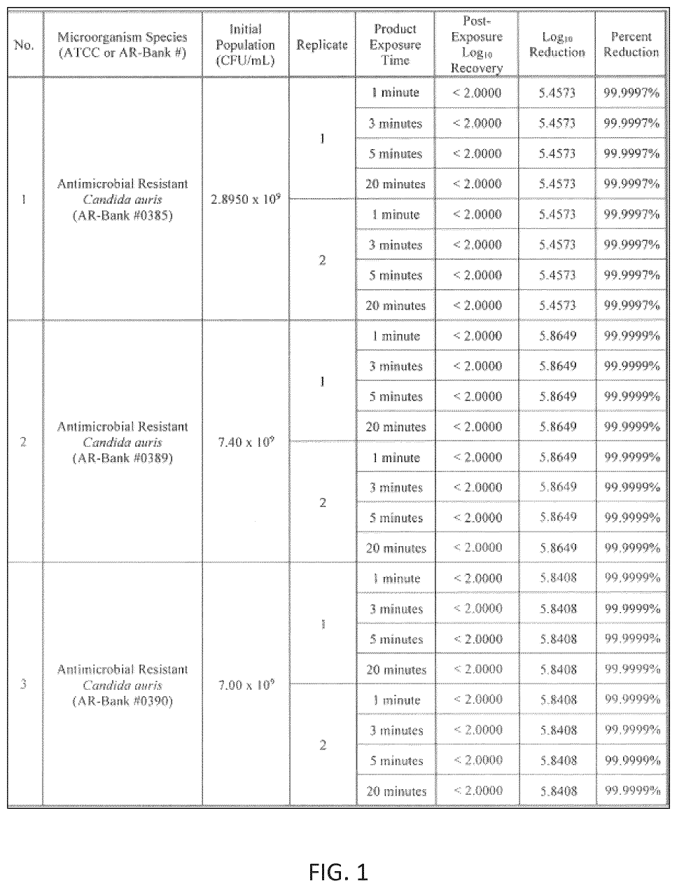

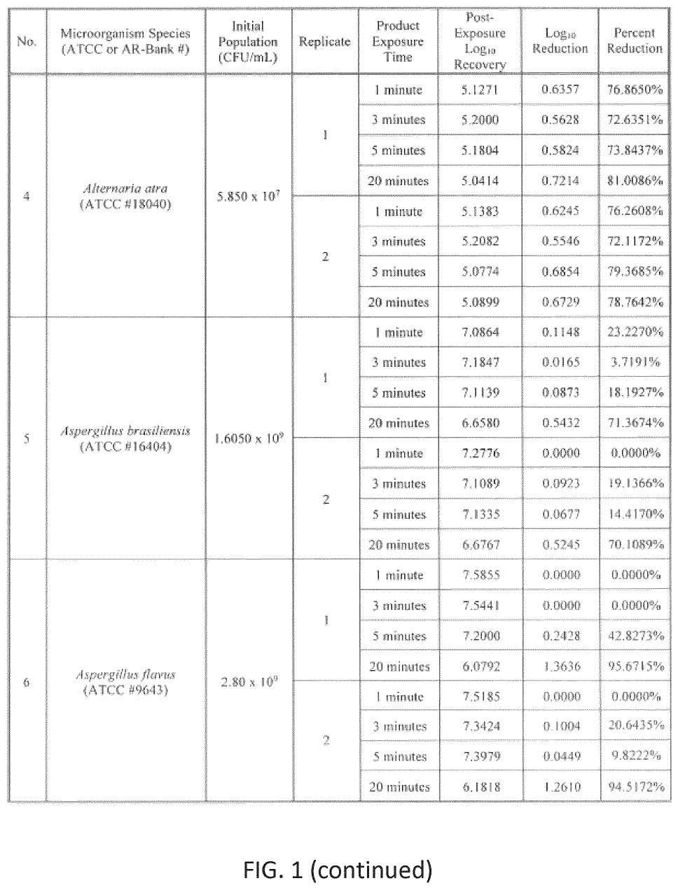

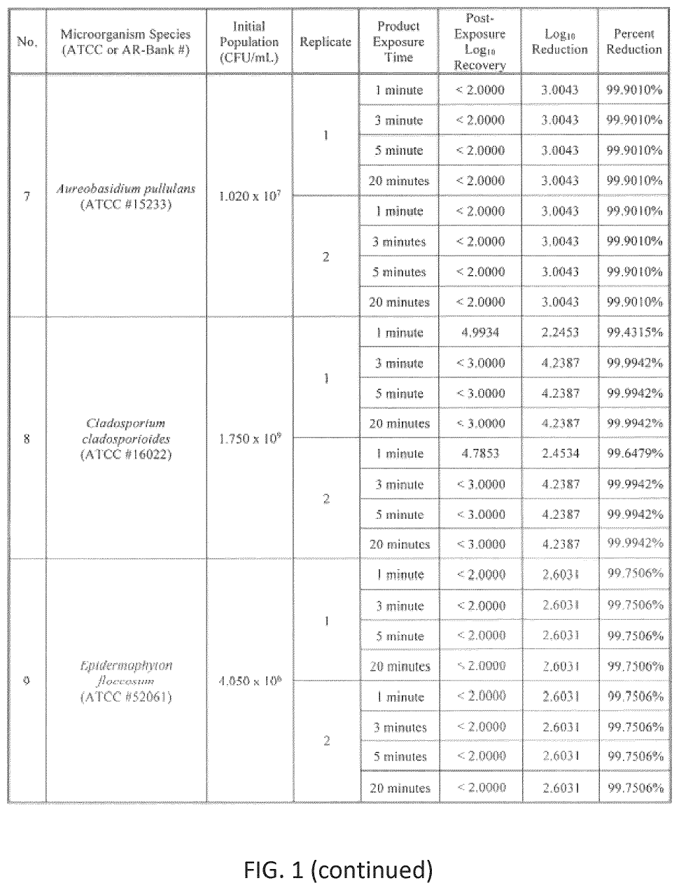

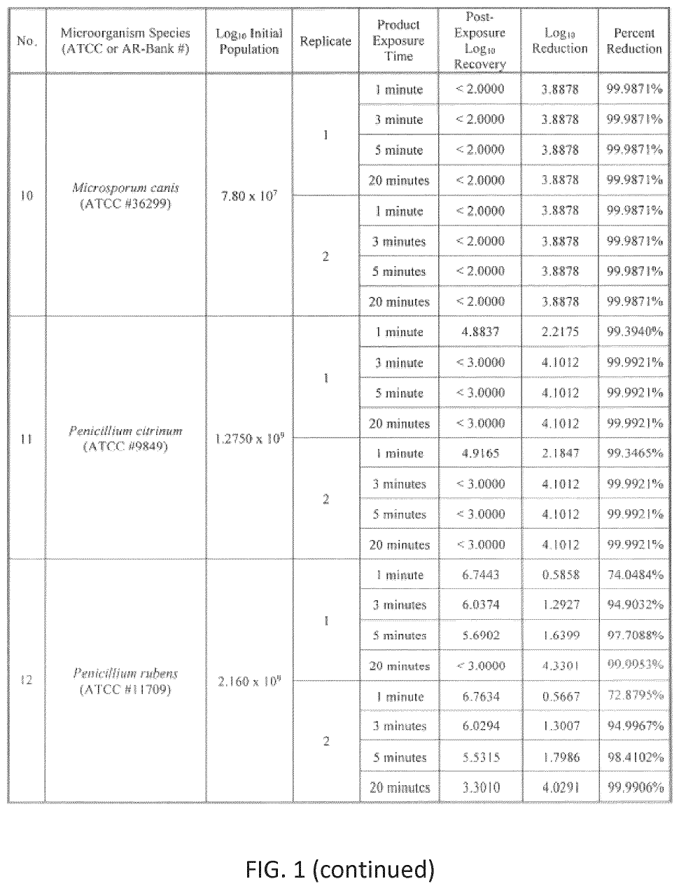

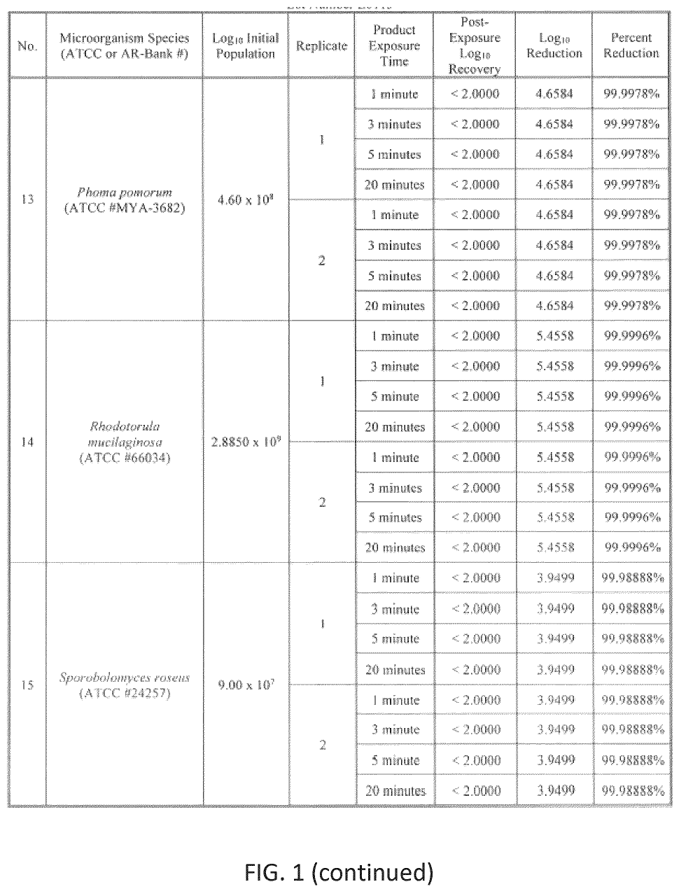

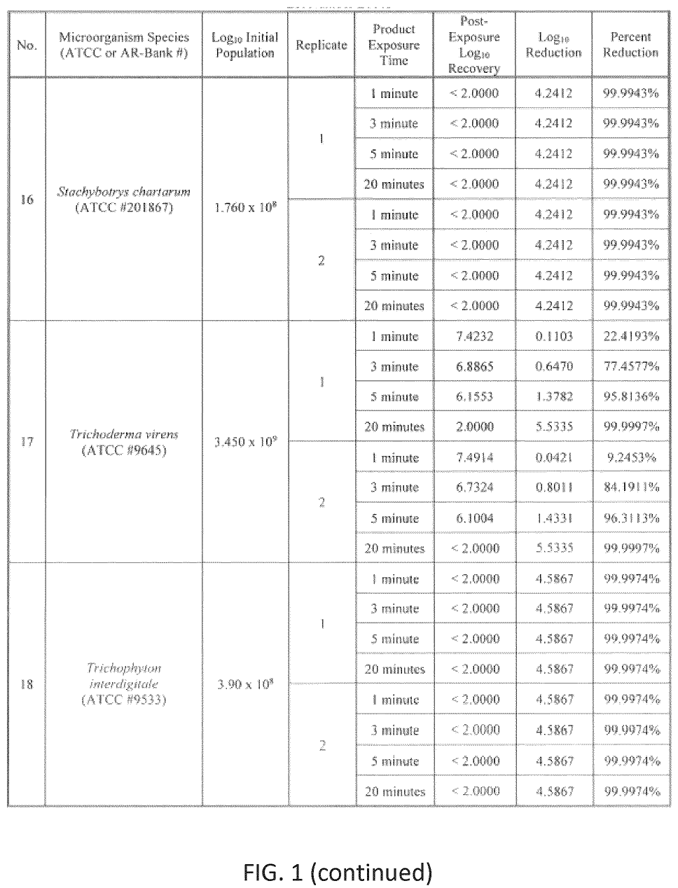

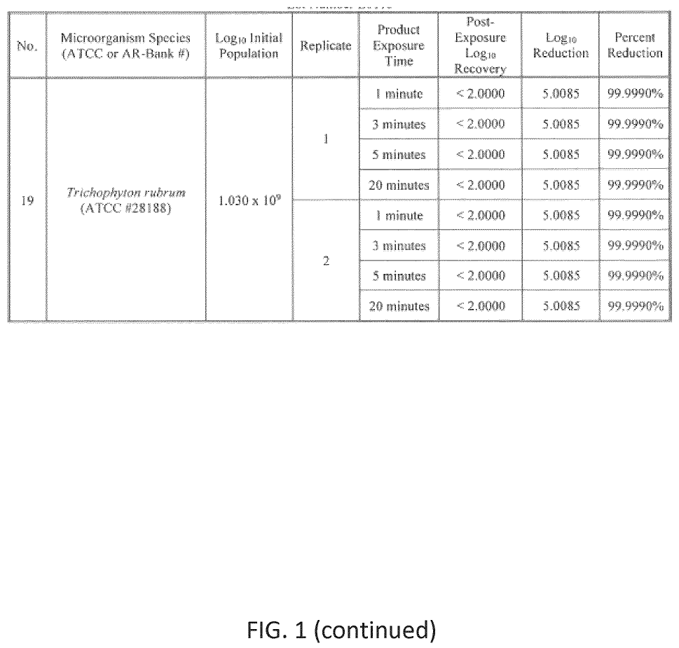

With reference to FIG. 1, Composition 1 reduced the fungal populations of 5 challenge fungal species by more than 5.0 log₁₀ following-minute exposures: The 5 species were antimicrobial Resistant Candida auris (AR-Bank #0385, AR-Bank #0389, and AR-Bank #0390), Rhodotorula mucilaginosa (ATCC #66034), and Trichophyton rubrum (ATCC #28188). The following three fungal species-Phoma pomorum (ATCC #MY A-3682), Stachybotrys chartarum (ATCC #201867) and Trichophyton interdigitate (ATCC #9533) were each reduced by more than 4.0 log 10 following 1-minute exposures. Aureobasidium pullulans (ATCC #15233), Microsporum canis (ATCC #36299), Sporobolomyces roseus (ATCC #24257), and Epidermophyton floccosum (ATCC #52061) were reduced by more than 2.6 log 10 following a 1-minute exposure. The population of Penicillium citrinum (ATCC #9849) and Cladosporium cladosporioides (ATCC #16022) were reduced by more than 4.1 log 10, following a 3-minute exposure to Composition 1. The population of Penicillium ruben. (ATCC #11709) and Trichoderma virens (ATCC #9645) were reduced by more than 4.3 log 10 following a 20-minute exposure. Aspergillus flavus (ATCC #9643) was reduced by 1.2 log 10 following a 20-minute exposure. The population of Aspergillus brasiliensis (ATCC #16404) and Alternaria atra (ATCC #18040) were reduced by <1.0 log 10, following a 20-minute exposure to Composition 1.

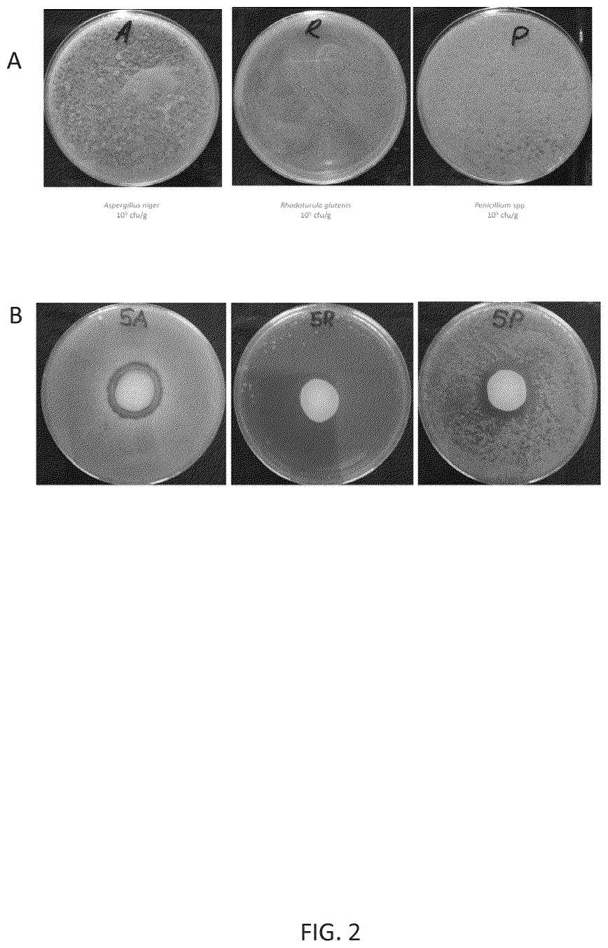

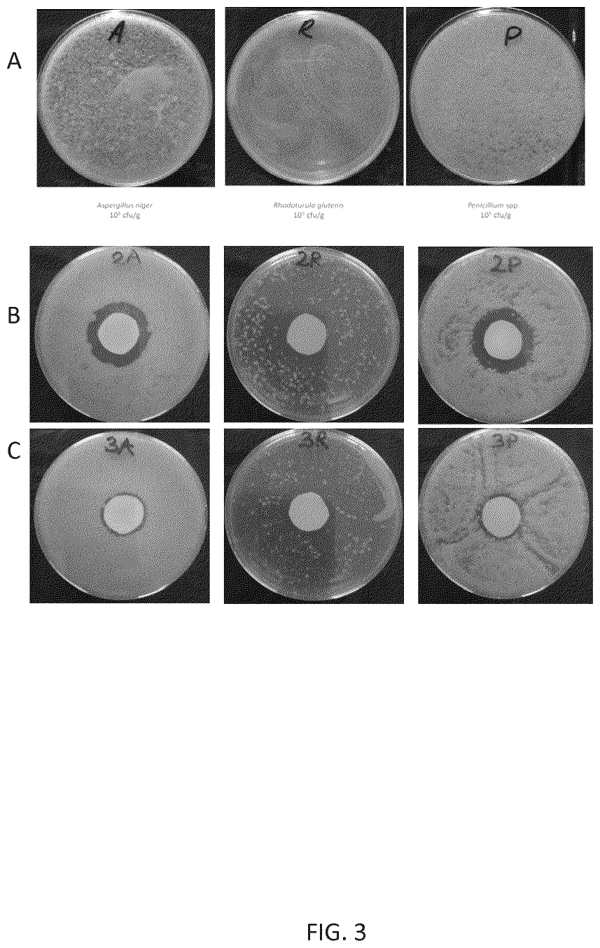

With reference to FIGS. 2 and 3, the anti-fungal activity of Composition 1 was further tested using a standard zone of inhibition disk assay. Rose Bengal with Chlorampheicol (DBRC) agar plates were seeded with 0.1 ml of Aspergillus niger (10⁵ cfu/g), Rhodotrula glutenis (10⁵ cfu/g) or Penicillum spp (10⁵ cfu/g) culture suspension. 1.5 cm Whatman paper disks treated with the anti-fungal agent was placed in the center of each plate except for the control plates. The plates were incubated under standard conditions at 25° C. for 3 days and the zone of inhibition assessed.

According to this well-established test, the amount of space under and around the substrate disk indicates the effectiveness of the tested composition in inhibiting the growth of the fungus in question. The effective composition(s) produce a ring of no fungal growth, while ineffective compositions show no change in the surrounding fungal concentration at all.

FIG. 2A shows untreated fungi. FIG. 2B shows zone of inhibition around a disk infused with Formulation 1. Activity against Aspergillus niger (10⁵ cfu/g), Rhodotrula glutenis (10⁵ cfu/g) and Penicillum spp (10⁵ cfu/g) was observed. The largest ring was observed for Rhodotrula glutenis.

For comparison the anti-fungal activities of a combination of 2-methyl-4-isothiazolin-3-one (0.5 wt % in water) and 1,2-benzisothiazol-3 (3H)-one (0.5 wt % in water), and that of 5-choloro-2-methyl-4-isothiazolin-3-one (0.5 wt % in water) against Aspergillus niger, Rhodotrula glutenis and Penicillum spp are shown in FIGS. 3B and 3C respectively. FIG. 3A shows untreated fungi.

Comparing the results of FIGS. 2 and 3 suggests that the efficacy of Composition A against Aspergillus niger, Rhodotrula glutenis and Penicillum spp was equal to or greater than the efficacy of the combination of 2-methyl-4isothiazolin-3-one (0.5 wt %) and 1,2-benzisothiazol-3 (3H)-one (0.5 wt %). and 5-choloro-2-methyl-4-isothiazolin-3-one.

### Example 4: Evaluation of In Vivo Anti-Fungal Activity

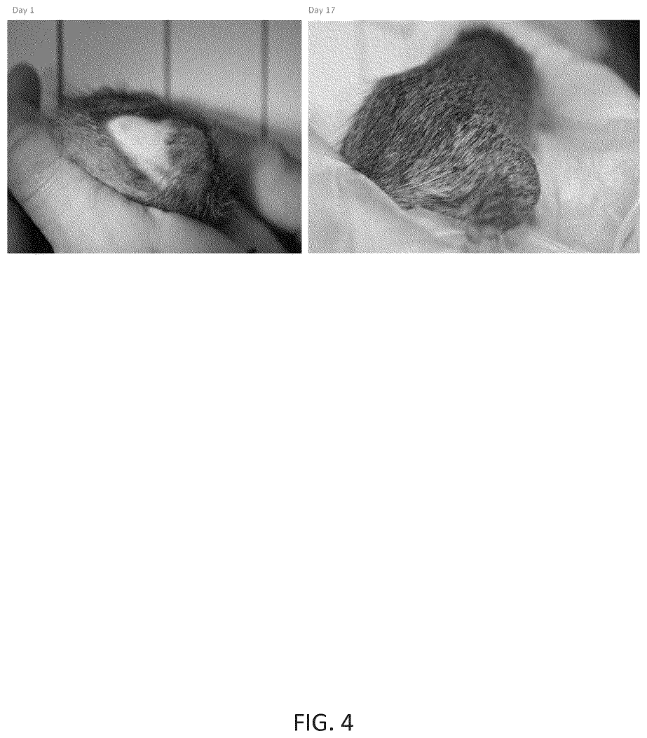

Referring to FIG. 4, affected areas of a degu mouse with mycosis were treated twice daily (12 hours apart) with a topical spray of formulation 2. After 17 days, visible signs of infection were resolved.

1. An anti-fungal composition comprising a combination of:
a) about 50% to about 70% of at least one unsaturated fatty acid having from 6 to 16 carbon atoms or a pharmaceutically acceptable salt thereof;
b) about 20% to about 35% at least one alpha-hydroxy acid or a pharmaceutically acceptable salt thereof; and
c) about 10% to about 20% at least one amine

2. The anti-fungal composition according to claim 1, comprising:
about 55% to about 65% of the unsaturated fatty acid;
about 25% to about 30% of the alpha-hydroxy acid; and
about 10% to about 15% of the amine.

3. The anti-fungal composition according to claim 1, comprising:
about 58% of the unsaturated fatty acid;
about 28% of the alpha-hydroxy acid; and
about 14% the amine.

4. The anti-fungal composition according to claim 1, wherein the unsaturated fatty acid is undecylenic acid or a pharmaceutically acceptable salt thereof.

5. The anti-fungal composition according to claim 1, wherein the alpha hydroxy acid is lactic acid.

6. The anti-fungal composition according to claim 1, wherein the amine is selected from ethylenediamine, N,N,-dimethylbutylamine, triethylamine or a combination thereof.

7. The anti-fungal composition according to claim 1, wherein the amine is an amino alcohol having the formula: 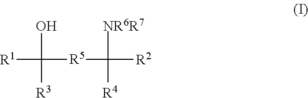
wherein R¹ and R³ are each independently H, alkyl, R² and R⁴ are each independently H, alkyl, or hydroxyalkyl; R⁵ is absent or is a C₁-C₆ alkylene; and R⁶ and R⁷ are independently H, C₁-C₆ alkyl or hydroxyalkyl.

8. The anti-fungal composition according to claim 7, wherein the amino alcohol is selected from monoethanolamine (MEA), triethanolamine (TEA), 2-amino-2-methyl-1-propanol, 2-amino-2-methyl-1,3-propanediol, 2-amino-2-ethyl-1,3-propanediol,2-dimethylamino-2-methylpropanol, dimethylglucamine, n-butyldiethanolamine, 2-butylamino ethanol, or a combination thereof.

9. The anti-fungal composition according to claim 1, wherein the composition is formulated as a solution, emulsion or dispersion in a suitable liquid medium.

10. (canceled)

11. The anti-fungal composition according to claim 9, wherein the liquid medium is water, an organic solvent, an alcohol or oil.

12. A pharmaceutical formulation comprising a composition as defined in claim 1, and a pharmaceutically acceptable carrier.

13. The pharmaceutical formulation according to claim 12, wherein said formulation is for topical administration or oral administration.

14.-16. (canceled)

17. A method of killing and/or inhibiting the growth and/or proliferation of fungus on a substrate comprising applying an effective amount of the anti-fungal composition as defined in claim 1.

18. (canceled)

19. A method of treating or preventing a fungal infection, or a disease or disorder associated therewith, in an mammal in need thereof, comprising administrating an effective amount of the anti-fungal composition as defined in claim 1.

20. The method according to claim 19, comprising administrating said composition in combination with one or more anti-fungal agent(s).

21.-22. (canceled)

23. A personal care product comprising the anti-fungal composition of claim 1.

24. A household product comprising the anti-fungal composition of claim 1.

25. A paper product comprising the anti-fungal composition of claim 1.

26. A plastic product comprising the anti-fungal composition of claim 1.
